(19) United States
(12) Patent Application Publication
(10) Pub. No.: US 2022/0047053 A1
(43) Pub. Date: Feb. 17, 2022

(54) DEVICE FOR DISPLAYING AND PACKAGING HAIRDRESSING INSTRUMENTS

(71) Applicant: SCPI, Chavanod (FR)

(72) Inventor: Sébastien Nicoud, Annecy-le-Vieux (FR)

(21) Appl. No.: 17/298,998

(22) PCT Filed: Dec. 3, 2019
(86) PCT No.: PCT/IB2019/060404

§ 371 Date: Jun. 2, 2021

(30) Foreign Application Priority Data
Dec. 4, 2018 (FR) 18 72308
Sep. 24, 2019 (FR) 19 10551

(51) Int. Cl. A45D 6/00; A45D 20/52; A46B 9/02; A46B 17/06; A61L 2/10

(52) U.S. Cl. CPC A45D 6/00; A45D 20/525; A46B 9/023; A46B 17/065; A61L 2/10; A61L 2202/122; A45D 2006/005; A46B 2200/104; A61L 2202/11; A61L 2202/14; A45D 2200/205

(57) ABSTRACT

A device for displaying and packaging hairdressing instruments, including a plurality of blow-dry rollers, at least one removable gripping handle to which one of the blow-dry rollers can be attached, and a storage housing. The storage housing includes a receiving compartment dimensioned to receive and contain the plurality of blow-dry rollers, a peripheral wall of the receiving compartment, a bottom wall of the receiving compartment, and a retractable top cover. Stabilizing devices are shaped to each hold one of the blow-dry rollers oriented along a respective substantially vertical axis and separated from the other rollers. An intermediate space contains an ultraviolet radiation source for directing and distributing ultraviolet radiation into the receiving compartment. Magnets are distributed on the inner face of the cover for retaining metal tools. An electrical safety switch actuated by the cover inhibits the supply of power to the ultraviolet radiation source when the cover is not in the closed position.

### TECHNICAL FIELD OF THE INVENTION

The present invention relates to devices intended to be used for the hair, more particularly for the hairdressing process known as “blow-drying” or “shaping”, and for cutting the hair.

Blow-drying is a technique for drying and shaping the hair that is well known to hairdressers, involving the combined action of a hand-held hairdryer and a substantially cylindrical hand brush. This hairdressing technique consists in working, lock by lock, on wet hair with the aid of a brush, a blow-dry roller, a comb or the fingers, while drying them with a hand-held hairdryer.

In this technique, the hairdresser can create volume in or curl a head of hair. For this purpose, they have to individually take a lock of wet hair, wrap it around the hand brush or the blow-dry roller, dry it with the hand-held hairdryer and wait for it to cool down, in order to achieve a shape determined by the diameter of the brush or of the blow-dry roller.

To make blow-drying easier, the document FR 3 018 032 A1 describes a kit comprising a plurality of blow-dry rollers, at least one removable gripping handle to which one of the blow-dry rollers can be fixed, and a storage case having a receiving compartment designed to receive and contain the blow-dry rollers and the gripping handle. The receiving compartment is delimited by a peripheral wall, a bottom wall, and a retractable upper lid. The bottom wall has rows of stabilizing housings. The blow-dry rollers are held in vertical position and, for this purpose, each have an end peg which fits in one of the stabilizing housings in the bottom wall. The storage case also comprises a lower compartment which is situated beneath said lower wall of the receiving compartment and contains an ultraviolet radiation source for directing ultraviolet radiation upwardly in the direction of the lower wall of the receiving compartment, this lower wall being transparent to the ultraviolet radiation so as to allow the ultraviolet radiation to pass through upwardly into the receiving compartment. A drawback of such a device is the weakness of the lower wall. Another drawback is that it proves difficult to disinfect the blow-dry rollers. Another drawback is the lack of safety of the device, since the ultraviolet radiation can reach the user of the device when the lid is open. Another drawback is the lack of means for holding tools other than the blow-dry rollers. Another drawback is the impossibility of effectively cleaning the interior of the device, several areas being difficult to reach.

On the other hand, the document US 2013/0078142 A1 describes an apparatus for sterilizing medical instruments using ultraviolet radiation. The apparatus comprises a parallelepipedal case provided with a lateral door for accessing an interior compartment bounded by a lower intermediate wall and an upper intermediate wall, both of which are transparent and allow ultraviolet radiation produced by two series of upper and lower lamps to pass through. The medical instruments to be sterilized are introduced into the interior compartment either enclosed in a sealed pouch or enclosed in a sealed kit having a transparent wall.

Such an apparatus is not suitable for easily and ergonomically treating hairdressing instruments of different shape and structure such as brushes, combs, scissors.

### DISCLOSURE OF THE INVENTION

According to a first aspect, an object of the present invention is in particular to design a device for improving the hygienic conditions, by automatic, effective and ergonomic disinfection of all hairdressing instruments such as brushes, combs, scissors, in particular by more effective disinfection of the bristles of the brushes or blow-dry rollers.

To achieve these aims and others according to the abovementioned first aspect, the invention proposes a device for displaying and packaging hairdressing instruments, comprising a storage case having:
a receiving compartment designed to receive and contain at least a plurality of blow-dry rollers and at least one removable gripping handle to which one of the blow-dry rollers can be fixed,
a receiving compartment bottom wall,
a plurality of stabilizing means, each designed to receive and retain on the bottom wall, by axial engagement and in a removable manner, one of the blow-dry rollers that is oriented along a respective axis substantially perpendicular to the receiving compartment bottom wall, and at a distance from the other blow-dry rollers,
a receiving compartment peripheral wall,
an upper opening, opposite the receiving compartment bottom wall,
an upper lid, which is movable between a closed position in which it closes off the upper opening and an open position in which it allows access to the receiving compartment through the upper opening, wherein:
said plurality of stabilizing means are distributed over the bottom wall in a single line along the peripheral wall, around an intermediate space,
an ultraviolet radiation source is disposed in the intermediate space and is linked to electrical connection means for connecting it to an electrical power source,
the ultraviolet radiation source comprises a base, which is fixed in the storage case and on which the holder of an ultraviolet radiation lamp is fitted, which is itself of elongate shape and held in the intermediate space,
all or some of the walls of the storage case are structured such that their inner face is reflective for the ultraviolet radiation generated by the ultraviolet radiation source.

By virtue of the ultraviolet radiation source being disposed in the intermediate space, and by virtue of the blow-dry rollers being disposed in a single line and in a vertical position at the periphery of the intermediate space when they are held on the stabilizing means, a large part of the ultraviolet radiation propagates directly from the ultraviolet radiation source to the face of the blow-dry rollers that is oriented toward the ultraviolet radiation source, and radially reaches said blow-dry rollers, thereby being able to penetrate through the entire thickness of the layer of bristles of the blow-dry rollers. Moreover, by virtue of the very same arrangements as mentioned above, the ultraviolet rays propagate uniformly toward all the blow-dry rollers and along the entire length of said blow-dry rollers. Moreover, by virtue of the very same arrangements as mentioned above, and by virtue of the reflection of the ultraviolet radiation by the reflective inner surfaces of the walls of the receiving compartment, a large part of the ultraviolet radiation propagates, after reflection, toward the blow-dry rollers and reaches them radially, thereby being able to pass through the entire thickness of the layer of bristles of the blow-dry rollers via the face that is oriented toward the peripheral wall of the receiving compartment. Such an arrangement improves the propagation of the ultraviolet radiation while minimizing the bulk of the device, such that the hygienic conditions are significantly improved.

According to one embodiment, the receiving compartment has a parallelepipedal overall shape, the peripheral wall having two longitudinal sides oriented in a longitudinal direction and two transverse sides oriented in a transverse direction; said plurality of stabilizing means are arranged in two rows of stabilizing means each disposed respectively along one of the longitudinal sides of the peripheral wall, respectively on either side of the intermediate space.

According to another embodiment, the stabilizing means may also be arranged along at least one of the transverse sides of the peripheral wall, around the intermediate space.

According to a second aspect, an object of the invention is to improve the hygienic conditions by it being very easy to clean the interior of the device.

To achieve these aims and others according to the abovementioned second aspect, the invention provides for it to be easy to remove the ultraviolet radiation source. Specifically, in the known devices, the presence of the radiation source prevents certain interior areas of the device from being reached to clean them.

To this end, according to one embodiment:
the ultraviolet radiation source comprises a base, which is fixed in the storage case and on which the holder of an ultraviolet radiation lamp is fitted,
the base is fixed in the storage case by reversible fixing means allowing it to be released and removed from the storage case.

Such an arrangement allows the user to easily remove the assembly for generating ultraviolet radiation and the electrical connection means thereof, in order to separately clean this assembly and then to easily clean the interior of the receiving compartment. This considerably improves the hygiene of the device.

According to a first variant of this embodiment:
the receiving compartment has a parallelepipedal overall shape, the bottom wall extending in a longitudinal direction and in a transverse direction, the peripheral wall having two longitudinal sides oriented in the longitudinal direction and two transverse sides oriented in the transverse direction,
said plurality of stabilizing means distributed over the bottom wall are arranged in two rows of stabilizing means each disposed respectively along one of the longitudinal sides, on either side of the intermediate space,
the base is removably fixed to the inner face of one transverse side of a peripheral wall, the reversible fixing means being able to comprise slide means allowing the engagement and disengagement of the base by simply moving it in relative translation with respect to the storage case,
the ultraviolet radiation lamp is itself elongate in said longitudinal direction in the intermediate space.

According to a second variant of this embodiment:
the base is fixed to a lower case of the storage case,
the ultraviolet radiation lamp is itself elongate in a direction substantially perpendicular to the bottom wall in the intermediate space. In practice, the lower case may be disposed beneath the bottom wall of the receiving compartment, this bottom wall having a hole through which the ultraviolet radiation lamp and the protective cover thereof pass. This further improves the propagation of the ultraviolet radiation radially toward the blow-dry rollers, over the entire height thereof, and the blow-dry rollers can be positioned all around the intermediate space.

According to a third aspect, an object of the invention is to effectively prevent the ultraviolet radiation from being able to reach the user.

To achieve these aims and others according to the abovementioned third aspect, the invention provides for the electrical connection means to comprise an electrical safety switch, which is actuated by the lid and designed to prevent the ultraviolet radiation source from being supplied with power when the lid is not in the closed position. Thus, it is necessary to close the lid in order to allow the generation of ultraviolet radiation, thereby avoiding accidental output of ultraviolet radiation when the case is open.

In this case, the lid can be rotationally hinged to an upper edge of the peripheral wall, and may advantageously comprise a peg that engages with the electrical safety switch at the end of closure of the lid in order to close the electrical safety switch counter to elastic return means that tend to return said electrical safety switch into an open position. Thus, the opening of the lid automatically prevents the production of ultraviolet radiation, while the closing of the lid automatically causes the generation of ultraviolet radiation. Hygiene is thus improved since there is no risk of the user forgetting to turn on the ultraviolet radiation source.

According to a fourth aspect, an object of the invention is to design means for effectively holding various hairdressing instruments inside the device, while making it easier to introduce and remove these instruments.

To achieve these aims and others according to the abovementioned fourth aspect, the lid may comprise magnets that are able to retain, on its inner face, instruments comprising a ferromagnetic material. By virtue of the magnets, the user can easily store the cutting scissors by pressing them with one hand against the magnets, and he can then remove them as desired by taking hold of them with one hand to take them away from the magnets. Moreover, the cutting scissors are disinfected effectively by virtue of the magnets disposed on the inner face of the lid: the cutting scissors can be kept easily in an open position by the magnets, which keeps them facing the ultraviolet radiation source. The open position of the scissors allows disinfection in the entire cutting region of the blades thereof.

According to a fifth aspect, an object of the invention is to provide the user with a degree of freedom in the arrangement of the brushes or blow-dry rollers in the device, in order to allow an optimal number of brushes of different diameters to be used.

To achieve these aims and others according to the abovementioned fifth aspect, the stabilizing means are structured so as to receive and retain any blow-dry roller of said plurality of blow-dry rollers. In this way, one and the same device can receive blow-dry rollers of different diameters, as chosen by the user, without the latter having to pay particular attention to finding the right location for fitting a blow-dry roller on a stabilizing means.

According to one embodiment, said stabilizing means may comprise a peg on which a distal-end opening of a blow-dry roller can be fitted and retained. Preferably, said peg has a cylindrical base portion of circular cross section that is joined to a tapered free-end portion. Such a peg structure is particularly easy to clean, thereby further improving hygiene.

According to one preferred embodiment, said stabilizing means may be in the form of flat plates of material that are capable of being attracted by a magnet and are each intended to have the distal end of a blow-dry roller, itself provided with a magnet, bearing on them. Said plates define a preferential positioning zone for a blow-dry roller or brush, and may be designed to cover an area greater than the cross-sectional area of the blow-dry rollers or brush, in order to allow a degree of freedom in the positioning of the blow-dry roller or brush.

According to a sixth aspect, an object of the invention is to design such a device such that it exhibits good stability in all use positions, in particular when the lid is open and carrying instruments.

To achieve these aims and others according to the abovementioned sixth aspect, the invention provides a lower case, which is attached and retained in a removable manner under the bottom wall of the receiving compartment and contains a ballast weight dimensioned to avoid any risk of the receiving compartment tipping when the lid is open and carrying instruments.

According to one embodiment, the ballast weight is formed by a base of the ultraviolet radiation source and by the electrical connection means, optionally with a complementary weight such as a metal plate.

According to another embodiment, the ballast weight is formed by a battery of accumulators and electrical circuits for the power supply of the ultraviolet radiation source by said accumulators and for recharging said accumulators from an external electrical energy source.

Because the receiving compartment contains a plurality of blow-dry rollers oriented along a substantially vertical axis perpendicular to the opening closed off by said at least one lid, the hairdresser can take hold, with one hand, of a first blow-dry roller by handling just the removable gripping handle, carry out the treatment of a first lock of hair, remove the handle to leave the blow-dry roller in place on the first lock of hair, take a second blow-dry roller from the receiving compartment with the aid of the removable gripping handle, position the second blow-dry roller on a second lock of hair, and so on. At the same time, the hairdresser can use his other hand to operate the hairdryer in order to gradually dry the locks disposed on the blow-dry rollers. It will be understood that the handling of the blow-dry rollers and the handle can be carried out with one hand, leaving the other hand free to operate the hairdryer.

According to one feature of the invention, the ultraviolet radiation lamp may advantageously be covered with a protective cover, which is transparent to the ultraviolet rays and is itself fitted in a removable manner to the base. In particular, a cylindrical protective cover made of quartz can be provided, which is particularly economical and mechanically strong, avoiding the risks of deterioration.

According to one feature of the invention, to achieve good quality of reflection of the ultraviolet radiation, the inner surfaces of the walls of the storage case may advantageously be formed entirely or partially from polished aluminum or aluminum alloy.

To multiply the directions of reflection, the surfaces made of polished aluminum or aluminum alloy may advantageously be embossed. At the same time, this avoids the deterioration that usually results from scratches on flat reflective surfaces, since the recessed surface portions maintain their reflective properties.

In the case of an ultraviolet radiation lamp that is itself elongate in said longitudinal direction in the intermediate space, a convex cylindrical reflective surface may advantageously be positioned between the bottom wall and the ultraviolet radiation lamp and be oriented with a longitudinal axis parallel to the longitudinal axis of the ultraviolet radiation lamp.

According to one feature of the invention, the lid may advantageously also comprise, on its inner face, one or more clips having perforated elastic jaws, which are able to each receive and retain a comb in an orientation extending along an axis generally parallel to a lid bottom.

As an alternative, one or more combs can be fitted in holders provided on the inner face of the peripheral wall of the storage case, freeing up the entire inner surface of the lid for fixing metal scissors.

The particular arrangement of the stabilizing means and of the ultraviolet radiation source makes it possible to optimize the number of hairdressing instruments made available and disinfected, for a given size of the display and packaging device. The user can thus have access to all of the blow-dry instruments they need during a blow-drying or cutting step, and the device containing these instruments can, for this purpose, be disposed on the console table usually located in front of the chair on which the customer to be treated is sitting, without the presence of the device representing a hindrance or an unacceptable obstacle between the customer and the mirror above the console table.

The user having access to all of the instruments he needs avoids the need to move around unnecessarily. Also, there is no risk of the instruments disposed in the device according to the invention falling onto the ground and being damaged, and the user does not have to bend down to pick them up. Moreover, the instruments disposed in the receiving compartment or on the cover are accessible at a slightly raised height, and so the user does not need to bend down or tilt his chest forward each time he needs to change instrument. These instances of moving around, bending down or tilting, which are repeated numerous times over the course of a day, are the origin of numerous musculoskeletal problems which the invention therefore makes it possible to avoid.

According to one feature of the invention, the blow-dry rollers preferably comprise a cylindrical cage that has a distal end and is provided at a proximal end with a fixing end piece that is able to receive and retain, in a removable manner, by axial engagement, a fixing end of the gripping handle.

In a first embodiment, the distal end of the cylindrical cage is open through a distal-end opening intended to cooperate with a stabilizing means in the form of a peg.

In this way, the structure of the blow-dry rollers is simple and inexpensive, in particular as regards the distal-end opening of the cylindrical cage. The fixing end piece is also simple and inexpensive, being able to be made of one piece attached and bonded to the cylindrical cage.

In a second embodiment, the distal end of the cylindrical cage is closed off by a magnet intended to cooperate with a stabilizing means in the form of a plate made of a material that is capable of being attracted by a magnet.

According to one feature of the invention, the gripping handle may be provided with locking and unlocking means designed such that the gripping handle can be fixed to and released from the blow-dry roller by the one hand of the user holding the gripping handle.

According to a first possibility, the locking and unlocking means are mechanical and may comprise two opposing lateral pushbuttons, the simultaneous depression of which counter to an elastic return means unlocks the fixing end of the handle in the fixing end piece of the blow-dry roller, and the releasing of which allows the fixing end of the handle to be locked in the fixing end piece of the blow-dry roller when the fixing end of the handle is simply inserted axially into the fixing end piece of the blow-dry roller.

According to another possibility, the locking means may comprise magnets and respective conical engagement parts of the fixing end piece and of the handle. The engagement and disengagement can then be effected without the user needing to operate a pushbutton.

Preferably, the receiving compartment is designed to receive and contain the greatest possible number of blow-dry rollers, for a given volume of the receiving compartment.

However, according to one feature of the invention, for good transmission of the ultraviolet radiation and ease of gripping the blow-dry rollers, provision could advantageously be made for the stabilizing means to be distributed so as to leave a free space around each of the blow-dry rollers positioned on the stabilizing means, for example a free space of about 1 cm.

In practice, a receiving compartment may be provided, in which the longitudinal sides of the peripheral wall have a longitudinal dimension of between about 25 and 30 cm, and the transverse sides of the peripheral wall have a transverse dimension of between about 15 and 25 cm.

According to one advantageous embodiment, the receiving compartment may have an interior length of 27 cm and an interior width of about 20 cm. As a result, it is possible to accommodate in the receiving compartment:
2 large blow-dry rollers having an outside diameter of about 70 mm,
2 blow-dry rollers having an outside diameter of about 60 mm,
2 blow-dry rollers having an outside diameter of about 50 mm,
and 2 small blow-dry rollers having an outside diameter of about 40 mm.

These dimensions and numbers of rollers are particularly well suited for allowing the hairdresser to carry out a blow-drying process, with the aid of a device according to the present invention, the dimensions of which have been minimized.

According to another aspect, the invention provides an assembly comprising a device having a storage case as defined above, a plurality of blow-dry rollers, and at least one removable gripping handle to which one of the blow-dry rollers can be fixed.

### BRIEF DESCRIPTION OF THE DRAWINGS

Further subjects, features and advantages of the present invention will become apparent from the following description of particular embodiments, given with reference to the appended figures, in which:

FIG. 1 is a perspective view of a storage case according to one embodiment of the present invention, the upper lid being closed;

FIG. 2 is a perspective view of the storage case in FIG. 1, the upper lid being open, and the receiving compartment not having any blow-dry rollers;

FIG. 3 is another perspective view of the storage case in FIG. 2, the receiving compartment not having any blow-dry rollers;

FIG. 4 is a top view of the open storage case according to the embodiment in FIGS. 2 and 3;

FIG. 5 is a view in transverse section on the plane A-A in FIG. 4;

FIG. 6 is a view in longitudinal section on the plane B-B in FIG. 4, the storage case being closed and containing blow-dry rollers;

FIG. 7 is a view in transverse section on the plane A-A in FIG. 4, the storage case being closed and containing blow-dry rollers;

FIG. 8 is a top view of the storage case in FIG. 7, which is open and contains blow-dry rollers;

FIG. 9 is a perspective view of the device in FIG. 8;

FIG. 10 illustrates a blow-dry roller and the gripping handle thereof, in an embodiment of the present invention in which the distal end of the roller is open, and in which the handle is retained by mechanical locking means;

FIG. 11 is a view in longitudinal section of the blow-dry roller in FIG. 10;

FIG. 12 is a view in longitudinal section of a blow-dry roller and the gripping handle thereof, in another embodiment of the present invention in which the distal end of the roller is open, and in which the handle is retained by magnetic locking means;

FIG. 13 is a perspective top view of a blow-dry roller and the gripping handle thereof, in another embodiment of the present invention in which the blow-dry roller distal end is provided with a magnet, and in which the handle is retained by magnetic locking means;

FIG. 14 is a view in longitudinal section of the blow-dry roller and of the handle according to FIG. 13;

FIG. 15 is a perspective view of a storage case according to another embodiment of the invention, having guide ribs for fixing the assembly for generating ultraviolet radiation;

FIG. 16 is a perspective view of an assembly for generating ultraviolet radiation that can be fitted to the guide ribs of the case in FIG. 15;

FIG. 17 is another perspective view of the assembly for generating ultraviolet radiation in FIG. 16;

FIG. 18 is a perspective view of the tubular protective cover protecting the lamp for generating ultraviolet radiation;

FIG. 19 is a perspective view of a storage case according to another embodiment of the present invention, the upper lid having been removed to reveal dampers in the hinge system thereof;

FIG. 20 is a perspective view on a larger scale illustrating a hinge damper of the storage case in FIG. 19;

FIG. 21 is a perspective view of a closed storage case according to another embodiment of the present invention, provided with a lower compartment;

FIG. 22 is a perspective view of the storage case in FIG. 21, the lid being open to reveal the vertically oriented ultraviolet radiation lamp;

FIG. 23 is another perspective view of the storage case in FIG. 21;

FIG. 24 is a top view of the storage case in FIG. 22;

FIG. 25 is a view in transverse section of the storage case in FIG. 22;

FIG. 26 is a view in longitudinal section of the storage case in FIG. 22, provided with blow-dry rollers;

FIG. 27 is a view in transverse section of the storage case in FIG. 22, provided with blow-dry rollers, themselves illustrated in section;

FIG. 28 is a top view of the storage case in FIG. 22, provided with blow-dry rollers;

FIG. 29 is a perspective view of the storage case in FIG. 22, the lid being open to reveal the vertically oriented ultraviolet radiation lamp, and the blow-dry rollers; and

FIG. 30 is a perspective view illustrating the lower casing carrying an ultraviolet radiation source.

### DESCRIPTION OF THE PREFERRED EMBODIMENTS

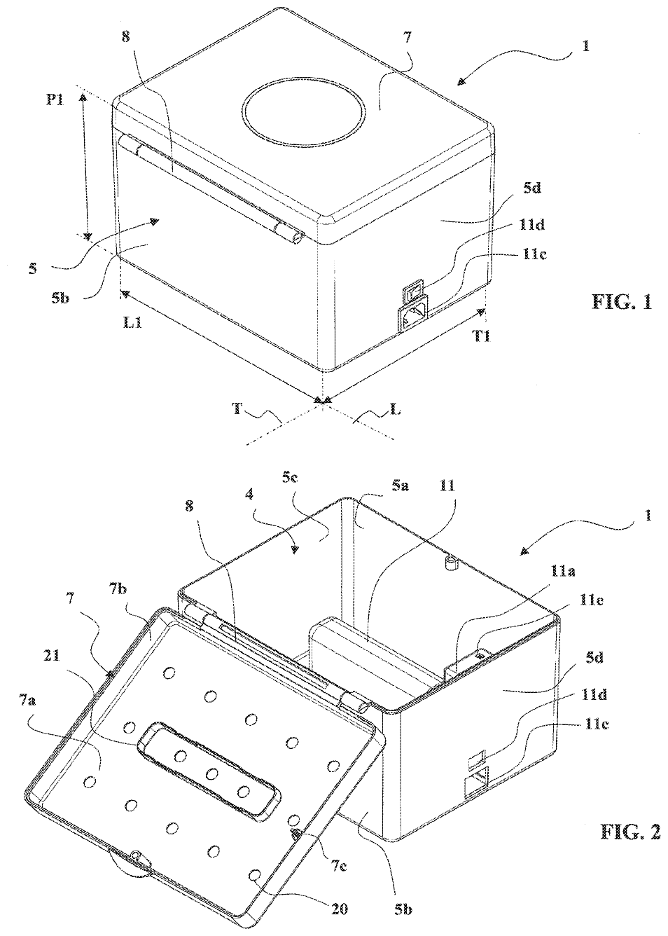

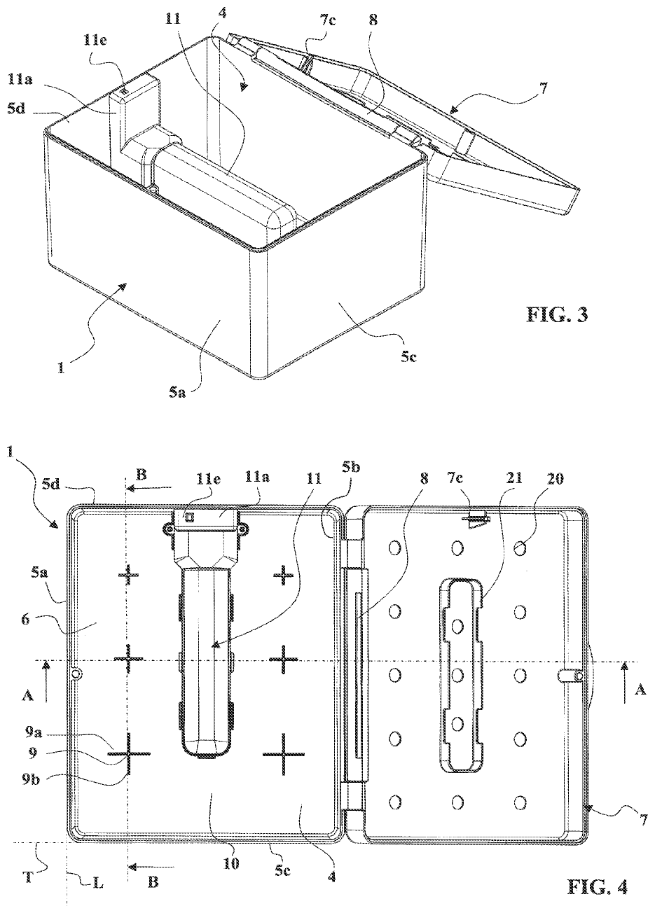

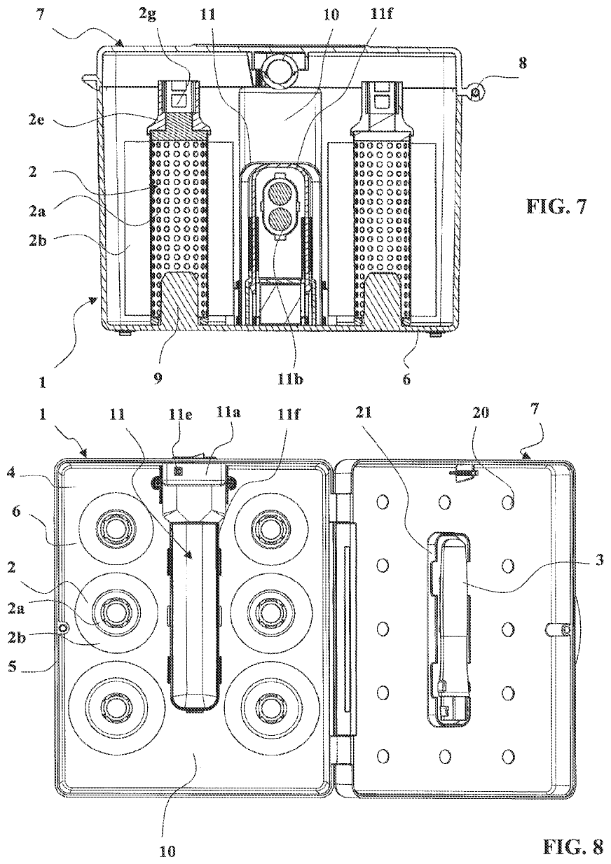

In the embodiment as illustrated in FIGS. 1 to 9, the device for displaying and packaging hairdressing instruments according to the invention comprises a storage case 1, a plurality of blow-dry rollers such as the roller 2, and at least one gripping handle 3.

The storage case 1 defines a receiving compartment 4, of parallelepipedal shape, designed to receive and contain said plurality of blow-dry rollers 2. By way of example, a receiving compartment 4 having a longitudinal dimension L1 of about 27 cm, a transverse dimension T1 of about 20 cm, and a depth dimension P1 of about 16 cm may be provided.

The receiving compartment 4 is bounded by a receiving compartment peripheral wall 5, a receiving compartment bottom wall 6, and a retractable upper lid 7, which is for example hinged to the upper edge of the peripheral wall 5 via a hinge 8.

A lid bottom 7a forms the upper wall of the storage case 1 and is joined to a peripheral rim 7b intended to form, in the closed position, the upper part of the peripheral wall 5 of the receiving compartment 4.

The bottom wall 6 extends in a longitudinal direction L and in a transverse direction T, such that the peripheral wall 5 comprises two longitudinal sides 5a and 5b oriented in the longitudinal direction L and two transverse sides 5c and 5d oriented in the transverse direction T.

The lid 7 can pivot about the hinge 8 between a closed position (FIGS. 1, 6, 7) in which it closes the receiving compartment 4 and a retracted position (FIGS. 2, 3, 4, 5, 8, 9) in which it allows free access to the interior space of the receiving compartment 4 through its upper opening.

The bottom wall 6 has a plurality of stabilizing means 9, which are distributed over the surface of the bottom wall 6 and are each designed to hold a blow-dry roller 2 and orient it along a respective axis I-I substantially perpendicular to the bottom wall 6 and at a distance from the other blow-dry rollers.

In order to simultaneously optimize the number of blow-dry rollers that the storage case 1 can contain and the effectiveness of disinfection ensured by the ultraviolet radiation, the stabilizing means 9 are arranged in two rows of stabilizing means each disposed respectively along one of the longitudinal sides 5a and 5b, on either side of an intermediate space 10 in which an ultraviolet radiation source 11 is disposed.

As can be seen in FIGS. 6 to 9, the blow-dry rollers 2 are oriented vertically and so their length has to be less than the depth L3 of the receiving compartment 4. In practice, the depth L3 of the receiving compartment 4 is therefore chosen such that it is greater than the length of the blow-dry rollers 2 that are intended to be used, in order that the presence of the blow-dry rollers 2 contained in the receiving compartment 4 does not prevent the lid 7 from being closed.

The ultraviolet radiation source 11 comprises a base 11a which is fixed to the inner face of the transverse side 5d and to which the holder of an ultraviolet radiation lamp 11b is fitted, which itself comprises two parallel tubes that are longitudinally elongate in the intermediate space 10. The ultraviolet radiation lamp 11b is covered by a protective cover 11f, which is transparent to the ultraviolet rays and is itself snap-fastened removably to the base 11a.

The ultraviolet radiation lamp 11b is supplied with electrical energy from an external electrical energy source via an electrical connection means comprising a power supply line terminated by a female power socket 11c that is accessible via the outer face of the transverse side 5d. The female power socket 11c is structured to receive a male plug of a power supply cord for connecting to an external electrical energy source. Connected in series in the power supply line, between the female power socket 11c and the ultraviolet radiation lamp 11b, is a first manually actuated electrical switch 11d, and likewise connected in series is an electrical safety switch 11e, which is itself actuated by the lid 7 and designed to prevent the ultraviolet radiation source 11 from being supplied with power when the lid 7 is not in the closed position. In practice, the lid 7 can comprise a peg 7c that protrudes from its inner face and engages with the electrical safety switch 11 e at the end of closure of the lid 7 in order to mechanically force the electrical safety switch 11e, counter to elastic return means, into the closed state, in which it establishes the power supply to the ultraviolet radiation lamp 11b.

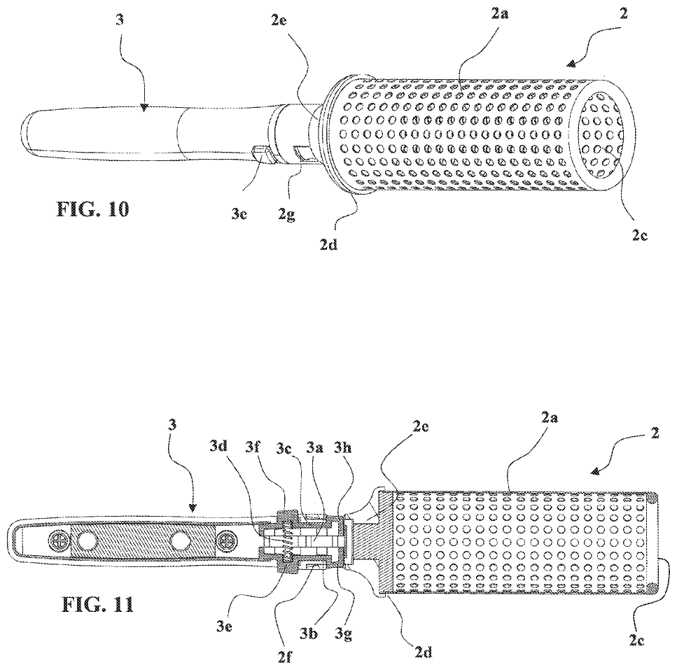

Reference is now made to FIGS. 10 and 11, which illustrate in more detail the structure of a blow-dry roller 2 and the cooperation thereof with the gripping handle 3, in an embodiment with mechanical locking.

In this embodiment, the blow-dry roller 2 comprises a cylindrical cage 2a carrying radially oriented bristles 2b, as is schematically illustrated in FIGS. 6 to 9. The cylindrical cage 2a has a distal-end opening 2c and a proximal end 2d to which a fixing end piece 2e is connected.

The fixing end piece 2e has an axial housing 2f designed to receive and retain, by axial engagement and in a removable manner, a fixing portion 3a of the gripping handle 3. Said fixing portion 3a of the gripping handle 3 is provided with locking and unlocking means comprising two respectively opposing lateral pushbuttons 3b and 3c, which are movable radially toward and away from one another by being pushed away from one another by an elastic return means 3d such as a helical compression spring as far as a locking position illustrated in FIG. 11.

Each of the lateral pushbuttons 3b and 3c comprises a proximal end having a respective radial protuberance 3e and 3f protruding from the lateral face of the gripping handle 3 to form a bearing surface that allows a user to depress the lateral pushbuttons 3b and 3c toward one another until they are in a pushed-together unlocking position. Each of the lateral pushbuttons 3b and 3c comprises a distal end having a respective radial locking peg 3g or 3h which, in the locking position, is engaged in a respective lateral locking hole 2g or 2h in the fixing end piece 2e to prevent the gripping handle 3 from being withdrawn from the cylindrical cage 2a. In the pushed-together unlocking position, the locking pegs 3g and 3h are both retracted into the axial housing 2f of the fixing end piece 2e, and allow the fixing portion 3a of the gripping handle 3 to freely slide axially in the axial housing 2f of the fixing end piece 2e, allowing the gripping handle 3 to be withdrawn from the cylindrical cage 2a or to be inserted into the cylindrical cage 2a.

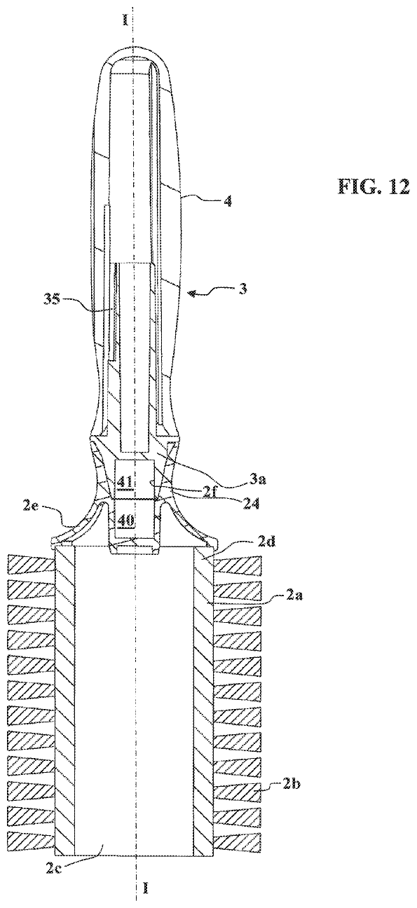

Reference is now made to FIG. 12, which illustrates in more detail the structure of a blow-dry roller 2 and the cooperation thereof with the gripping handle 3, in an embodiment with magnet locking.

In this embodiment, the blow-dry roller 2 has the same structure with a cylindrical cage 2a, bristles 2b, a distal-end opening 2c, and a proximal end 2d.

A fixed end piece 2e is attached to the proximal end 2d and comprises an axial housing 2f designed to receive and retain, by axial engagement and in a removable manner, a fixing portion 3a of the gripping handle 3. The fixing portion 3a and the axial housing 2f have a frustoconical shape as illustrated. A first magnet 40 is fixed in the bottom of the housing 2f. A second magnet 41 is fixed to the free end of the fixing portion 3a, and oriented to attract the first magnet 40.

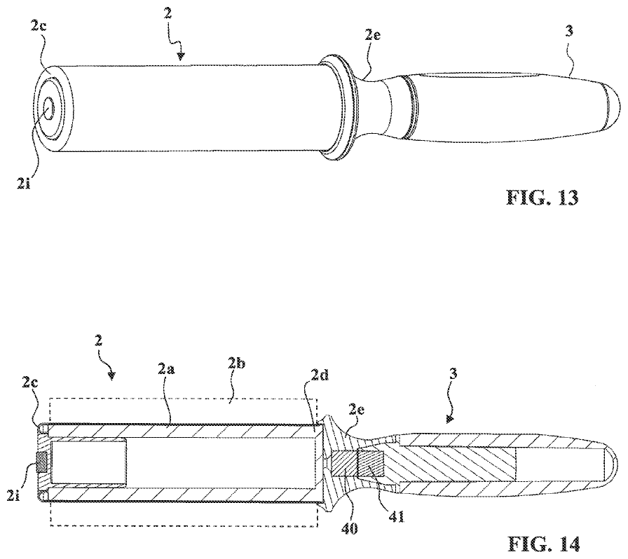

Reference is now made to FIGS. 13 and 14, which illustrate in more detail the structure of a blow-dry roller and the cooperation thereof with the gripping handle 3, again in an embodiment with magnet locking, but in which the distal-end opening 2c is closed off by a positioning magnet 2i.

In this embodiment, the blow-dry roller 2 has the same structure with a cylindrical cage 2a, bristles 2b, a proximal end 2d, while the distal-end opening 2c is closed off by the positioning magnet 2i. The fixing end piece 2e and the handle 3 have the same structure as in the previous embodiment, for locking by magnets 40 and 41.

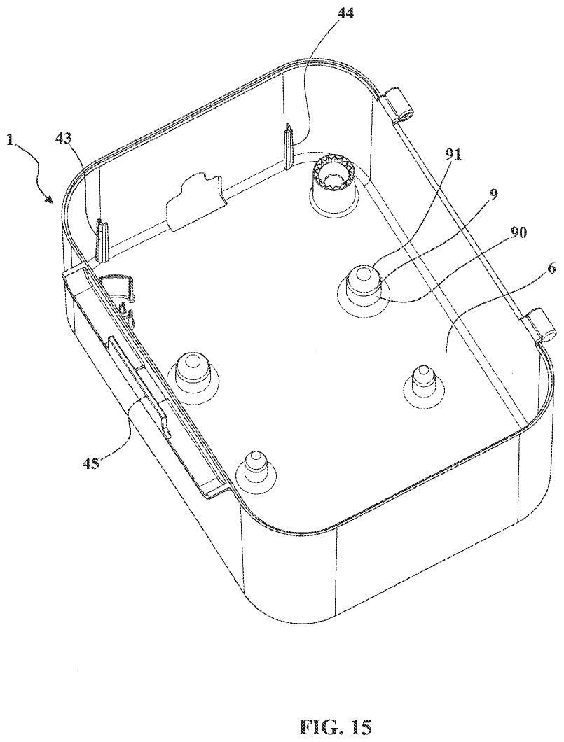

FIG. 15 illustrates a casing body 1 according to another embodiment of the invention. In this case, one of the transverse walls has two slides 43 and 44 on its inner face, which are oriented perpendicularly to the bottom wall 6. The upper longitudinal edge which does not have the hinge is provided with a holder 45 for receiving and retaining a cellphone that the hairdresser can use to display information relating to the customer whose hair is currently being styled.

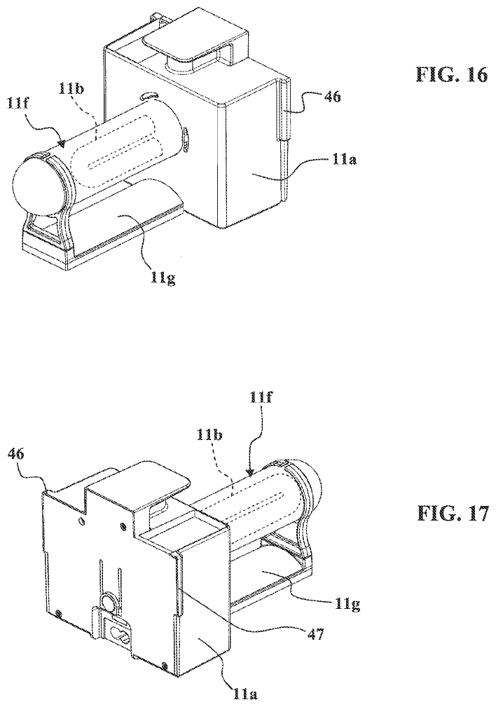

FIGS. 16 and 17 illustrate, in perspective another embodiment of the base structure 11 a and the ultraviolet radiation lamp 11b, with lateral ribs 46 and 47 that are able to cooperate with the slides 43 and 44 to allow the fitting of the base 11a by simply sliding it vertically in the direction of the bottom wall 6 and to allow the base 11a to be pulled by being slid in the other direction. Also apparent, in these figures, is the protective cover 11f of the ultraviolet radiation lamp. Also apparent is a convex cylindrical reflective surface 11g, which is positioned between the bottom wall 6 and the ultraviolet radiation lamp 11b and oriented with a longitudinal axis parallel to the longitudinal axis of the ultraviolet radiation lamp 11b.

The inner surfaces of the storage case 1 are reflective, making it possible to distribute the ultraviolet radiation in the useful region of the receiving compartment 4. For this purpose, when the walls are not made of a material that is itself reflective, the peripheral wall 5, the bottom wall 6 and the lid 7 of the receiving compartment 4 are provided with a respective reflective inner layer, for example a metal layer made of polished aluminum, preferably of polished and/or embossed aluminum.

The outer walls of the storage case 1, i.e. the lid 7, the peripheral wall 5 and the bottom wall 6, may be opaque, or covered with an opaque external layer that has a solid color or can be provided with a decoration or marking.

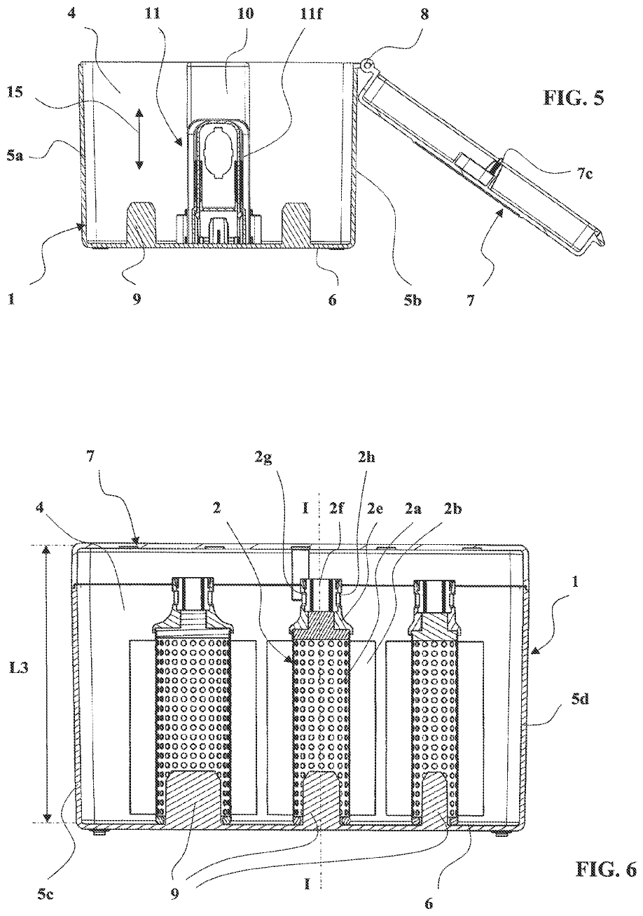

FIGS. 4 and 5 show an embodiment of the stabilizing means 9, each in the form of a peg on which the distal-end opening 2c of the cylindrical cage 2a of a blow-dry roller 2 can be fitted and retained.

One particular form of such a peg comprises two crossed walls 9a and 9b, perpendicular to the bottom wall 6. Optionally, provision may be made for the walls 9a and 9b to bear forcibly against the inner surface of the cage 2a, causing a slight elastic deformation of the cage 2a in order for it to be held in position better in the storage case 1.

Another, more advantageous, form of such a peg is illustrated in FIG. 15: consider, for example, the peg 9, which can be seen to be formed of a cylindrical base portion 90 of circular cross section that is joined to a tapered free end portion 91.

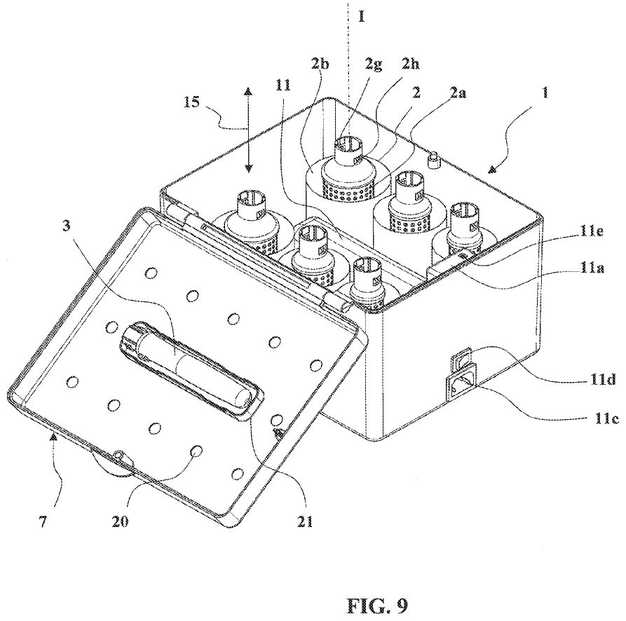

The vertical orientation of the blow-dry rollers 2 is advantageous in that it makes it possible to maximize the number of blow-dry rollers 2 that can be disposed in the receiving compartment 4, while making it easier to take hold of each blow-dry roller 2 with a gripping handle 3 without disturbing the positioning of the other adjacent rollers. The gripping handle 3 can be handled and moved easily using vertical movements in translation as illustrated by the double arrow 15 (FIG. 9), in order to introduce a blow-dry roller 2 into or remove it from the receiving compartment 4 when the lid 7 is open.

In the embodiment illustrated in FIGS. 2, 4, 8 and 9, the lid 7 comprises, on its inner face, a plurality of magnets 20 that are able to retain metal tools on said inner face of the lid 7. Preferably, the surface of the magnets 20 is flush with the inner face of the lid 7. It is thus possible to position one or more hair-cutting scissors on the inner face of the lid 7, these scissors preferably being in an open position, the magnets 20 keeping them pressed in position on said inner face of the lid 7 after the storage case 1 has been closed.

The central region of said inner face of the lid 7 comprises an elongate housing 21 with elastic lateral tabs for receiving and retaining the gripping handle 3 mechanically, said gripping handle 3 thus being able to be stored when it is not being used and to be disinfected by the action of the ultraviolet radiation when the storage case 1 is closed.

According to one embodiment of the invention, the lid 7 may also comprise, on its inner face, one or more comb-retaining clips, with which one or more combs can each be retained on the inner face of the lid 7. For example, the clip may comprise a flat generally parallelepipedal clip body oriented on a plane generally perpendicular to the peripheral rim 7b and to the bottom 7a of the lid 7, a first side of which is fixed to the peripheral rim 7b of the lid 7, and comprising an open central slot on a second side opposite the first side and on a third side away from the lid bottom 7a. The central slot separates two clip jaws from one another, these being themselves perforated. The clip body is made of a resilient material, for example a plastics material, which allows the clip jaws to be moved apart counter to an elastic return force that returns them into the vicinity of one another in the absence of loading.

As a result, a user can force-fit a comb into the central slot of the clip, the comb preferably having an elongate orientation along an axis generally parallel to the lid bottom 7a. The holes allow the penetration of the ultraviolet radiation for acting on the comb retained in position in the clip.

Two clips that are oriented parallel to one another may be provided.

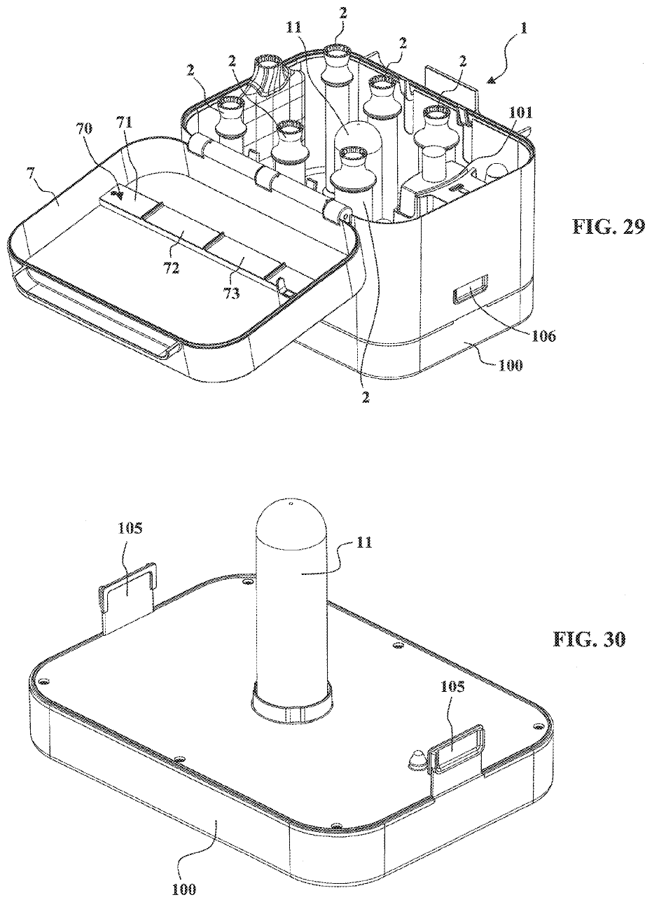

According to a preferred feature of the present invention, illustrated in the embodiment described with respect to FIGS. 22 and 29, the inner face of the lid 7 comprises at least one strip 70 having rectangular magnets 71, 72, 73 arranged in a line. In this case, the inner face of the lid 7 does not have clips or a housing for receiving and retaining the handle 3. Conversely, the handle 3 itself has a magnet or a ferromagnetic material 35 (FIG. 12) in order to be retained by magnetic attraction against the inner face of the lid 7.

Advantageously, the receiving compartment 4 can be designed to receive and contain all of the blow-dry rollers that the hairdresser may need during a blow-drying process, i.e. two large blow-dry rollers having an outside diameter of about 70 mm, two blow-dry rollers having an outside diameter of about 60 mm, two blow-dry rollers having an outside diameter of about 50 mm, and two small blow-dry rollers having an outside diameter of about 40 mm.

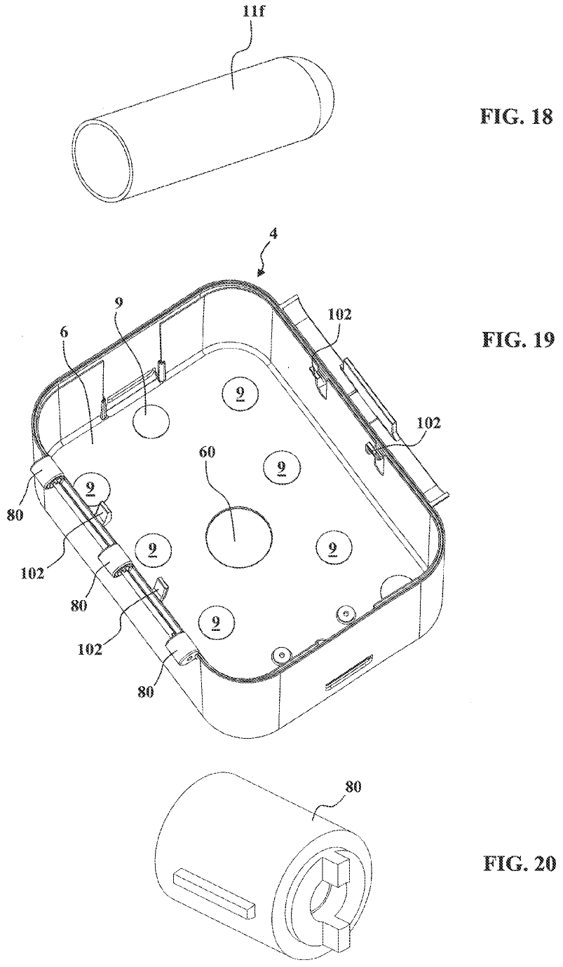

In the embodiment illustrated in FIG. 18, the protective cover 11f of the ultraviolet radiation lamp is a blind tube made of quartz, designed to fit around the ultraviolet radiation lamp, being retained in a removable manner on the base 11a.

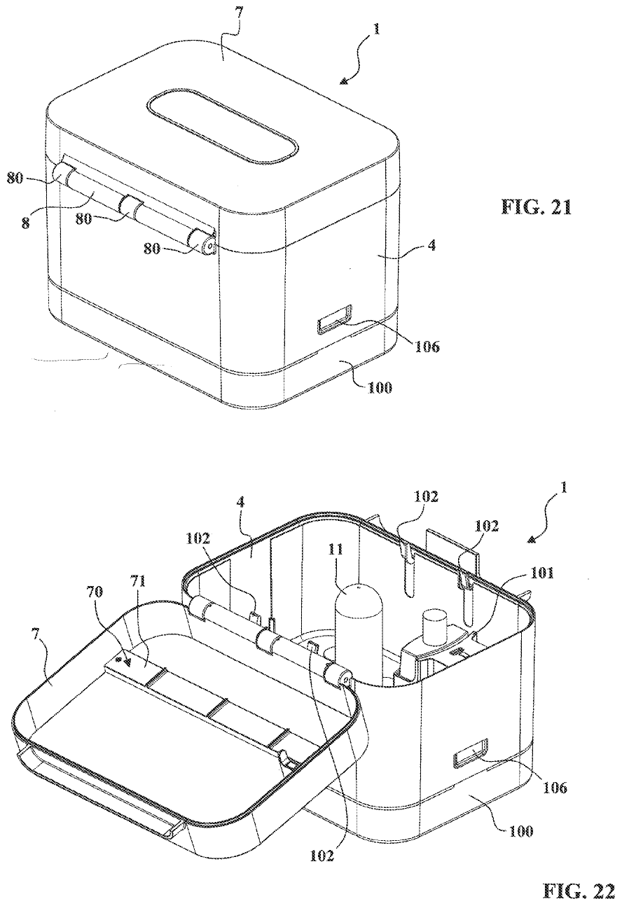

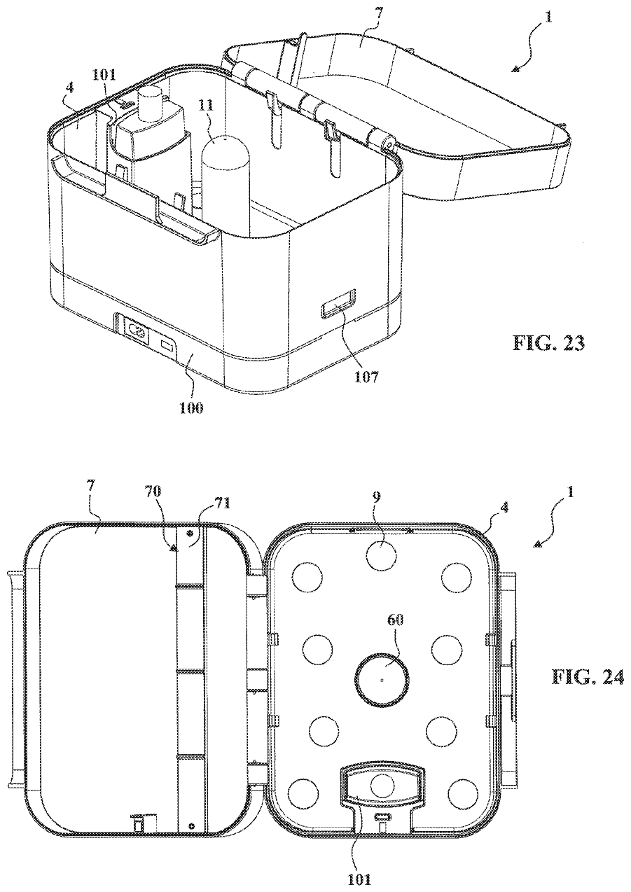

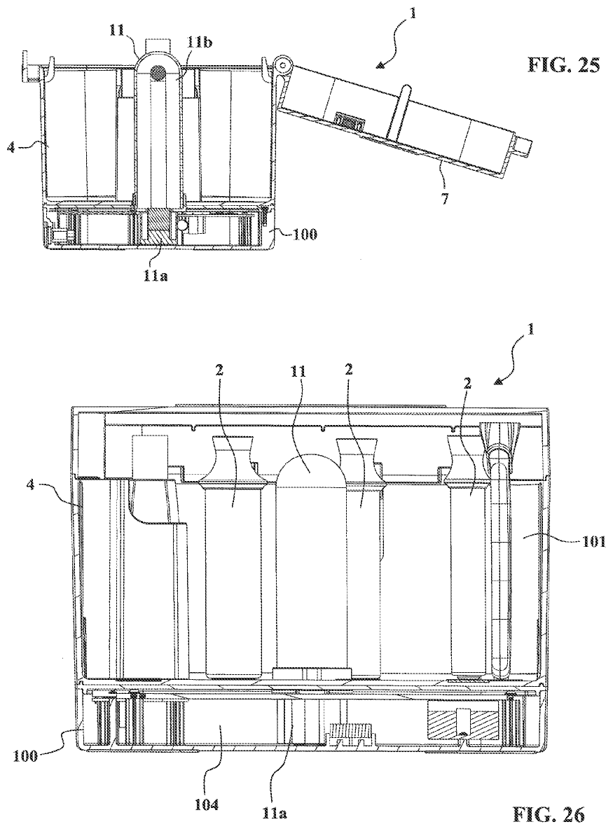

Reference is now made to the embodiment in FIGS. 19 to 30. This embodiment incorporates various different features of the present invention.

First of all, FIGS. 19 to 21 illustrate a hinge 8 provided with rotary dampers 80, illustrated on a larger scale in FIG. 20, for preventing the lid 7 from stopping too quickly in the open position and thus causing the receiving compartment 4 to tip.

Next, in FIG. 19, consideration should be given to the bottom wall 6 on which the stabilizing means 9 are made of plates made of a material that is able to be attracted by a magnet, for example made of ferromagnetic material, said plates being distributed in an appropriate manner to each receive a brush or blow-dry roller 2 according to the embodiment in FIGS. 13 and 14 with a positioning magnet 2i which cooperates with the stabilizing means 9 to keep the brush or blow-dry roller 2 in a vertical position on the bottom wall 6.

Next, in this same FIG. 19, there is, in the central part of the bottom wall 6, a hole 60 through which the ultraviolet radiation source 11 is intended to pass, as will be seen in the following figures.

In this embodiment in FIGS. 19 to 30, the device also has a lower case 100, attached and retained in a removable manner beneath the bottom wall 6 of the receiving compartment 4. The base 11a is fixed in said lower case 100 of the storage case 1. The ultraviolet radiation lamp 11b is itself elongate in a direction substantially perpendicular to the bottom wall 6, and passes through said hole 60 in the bottom wall 6 in order to be inserted with its protective cover in the intermediate space 10. This further improves the propagation of the ultraviolet radiation radially toward the blow-dry rollers 2, over the entire height thereof, and the blow-dry rollers 2 can be positioned all around the intermediate space 10.

In this embodiment in FIGS. 19 to 30, the lower case 100 may also serve as a stabilizing means, preventing the possible tipping of the device during the opening of the lid 7 containing relatively heavy tools such as metal scissors.

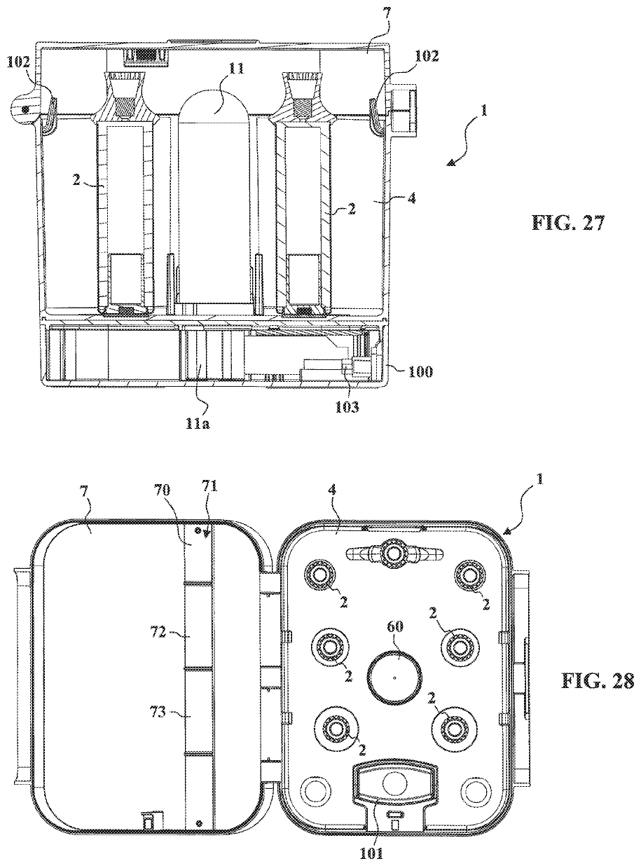

Stabilization can be ensured by a ballast weight formed by elements contained in the lower case 100, for example the base 11 a and the connection means 103 (FIG. 27) of the ultraviolet radiation lamp 11b, and/or a complementary weight, and/or a battery of accumulators 104 (FIG. 26) intended to supply said ultraviolet radiation lamp 11b with power while being itself rechargeable from an external electrical energy source.

Preferably, the lower case 100 is easily removable to make it easier to clean the device. For example, as can be seen in FIG. 30, the lower case 100 has two tongues 105 that are snap-fitted in corresponding holes 106 and 107 in the peripheral wall of the receiving compartment 4.

In the embodiment in FIGS. 19 to 30, there is also illustrated an optional feature consisting of a housing 101 intended to receive and retain a bottle of disinfectant liquid that the user can use to complete the disinfection of the tools if he deems this necessary. Also illustrated are tongues 102 intended to receive and retain one or more combs, avoiding the use of clips in the inner face of the lid 7.

To be used, the device according to the invention comprises a storage case 1 as defined above, a plurality of blow-dry rollers 2, and at least one removable gripping handle 3 to which one of the blow-dry rollers 2 can be fixed. The blow-dry rollers 2 comprise a cylindrical cage 2a provided at a proximal end 2d with a fixing end piece 2e that is able to receive and retain, by axial engagement and in a removable manner, a fixing portion 3a of the gripping handle 3.

When the devices illustrated in the figures are used, the hairdressers can carry out a blow-drying process using the sequence of the following steps: they take the gripping handle 3, they open the lid 7 of the storage case 1, they vertically fit the fixing portion 3a of the gripping handle 3 in the fixing end piece 2e of a first blow-dry roller 2, they fix the first blow-dry roller 2 to the gripping handle 3 by the action of the magnets 40 and 41 or by engaging the radial locking pegs 3g and 3h in the lateral locking holes 2g and 2h, they remove the assembly formed by the first blow-dry roller 2 and the gripping handle 3. They can then wrap a first lock of hair around the first blow-dry roller 2, and subject said first lock to a current of hot air produced by a hairdryer. The hairdresser thus holds the hairdryer in one hand and the gripping handle 3 and the first blow-dry roller 2 in the other hand.

During the treatment of the first lock of hair, the hairdresser may find it advantageous to work with the blow-dry roller 2—gripping handle 3 assembly, which forms a brush around which the first lock of wet hair is wrapped. During heating using the hairdryer, the brush thus formed makes it possible to wind and unwind the first lock of hair, accelerating the drying. The brush also makes it possible to smooth the hair by sliding to some extent.

After drying of the first lock of hair has been started, the hairdresser can remove the gripping handle 3 from the first blow-dry roller 2, which remains in place on the first lock of hair until the latter has completely dried and cooled down. On account of the absence of the gripping handle 3, the first blow-dry roller 2 remains balanced and allows a long drying process, which properly cools down the lock of hair without deforming it. The end of drying advantageously occurs away from the hairdryer, this improving the sheen of the hair.

During the end of drying and the cooling-down of the first lock of hair, the hairdresser can thus, without waiting, start the same operation again in order to take a second blow-dry roller 2 present in the storage case 1, in order to place this second blow-dry roller 2 in the head of hair and wrap a second lock of hair around this second blow-dry roller 2, and so on.

After a lock of hair has completely cooled down and dried, the hairdresser can refit the gripping handle 3 on the blow-dry roller 2 wound around said lock of hair, he removes the gripping handle 3—blow-dry roller 2 assembly from said lock of hair, he introduces the blow-dry roller 2 held by the gripping handle 3 into the receiving compartment 4 by engaging its distal end 2c over one of the stabilizing means 9, and he removes the gripping handle 3 from the receiving compartment 4.

The hairdresser also automatically carries out the disinfection of the blow-dry rollers 2 and of the hairdressing tools. For this purpose, once the blow-dry rollers 2 and other tools are disposed in the receiving compartment 4, he closes the lid 7, thereby closing the electrical safety switch 11e in order to supply the ultraviolet radiation source 11 with power and thus subject the blow-dry rollers 2 and other tools to ultraviolet radiation.

The present invention is not limited to the embodiments that have been explicitly described, but includes various variants and generalizations that fall within the scope of the following claims.

1-16. (canceled)

17. A device for displaying and packaging hairdressing instruments, comprising a storage case having:
a receiving compartment designed to receive and contain at least a plurality of blow-dry rollers and at least one removable gripping handle to which one of the blow-dry rollers can be fixed,
a receiving compartment bottom wall,
a plurality of stabilizing means, each designed to receive and retain on the bottom wall, by axial engagement and in a removable manner, one of the blow-dry rollers that is oriented along a respective axis (I-I) substantially perpendicular to the receiving compartment bottom wall, and at a distance from the other blow-dry rollers,
a receiving compartment peripheral wall,
an upper opening, opposite the receiving compartment bottom wall,
an upper lid, which is movable between a closed position in which it closes off the upper opening and an open position in which it allows access to the receiving compartment through the upper opening,
wherein:
said plurality of stabilizing means are distributed over the bottom wall in a single line along the peripheral wall, around an intermediate space,
an ultraviolet radiation source is disposed in the intermediate space and is linked to electrical connection means for connecting it to an electrical power source, t
he ultraviolet radiation source comprises a base, which is fixed in the storage case and on which the holder of an ultraviolet radiation lamp is fitted, which is itself of elongate shape and held in the intermediate space,
all or some of the walls of the storage case are structured such that their inner face is reflective for the ultraviolet radiation generated by the ultraviolet radiation source.

18. The device as claimed in claim 17, wherein the ultraviolet radiation lamp is covered with a protective cover, which is transparent to the ultraviolet rays and is itself fitted in a removable manner to the base.

19. The device as claimed in claim 17, wherein the inner surfaces of the walls of the storage case are formed entirely or partially from polished aluminum or aluminum alloy.

20. The device as claimed in claim 19, wherein the surfaces made of polished aluminum or aluminum alloy are also embossed.

21. The device as claimed in claim 17, wherein:
the receiving compartment has a parallelepipedal overall shape, the bottom wall extending in a longitudinal direction (L) and in a transverse direction (T), the peripheral wall having two longitudinal sides oriented in the longitudinal direction (L) and two transverse sides oriented in the transverse direction (T),
said plurality of stabilizing means distributed over the bottom wall are arranged in two rows of stabilizing means each disposed respectively along one of the longitudinal sides, on either side of the intermediate space,
the base is fixed to the inner face of one transverse side of a peripheral wall,
the ultraviolet radiation lamp is itself elongate in said longitudinal direction (L) in the intermediate space.

22. The device as claimed in claim 21, wherein a convex cylindrical reflective surface is positioned between the bottom wall and the ultraviolet radiation lamp and oriented with a longitudinal axis parallel to the longitudinal axis of the ultraviolet radiation lamp.

23. The device as claimed in claim 17, wherein:
the base is fixed in a lower case of the storage case,
the ultraviolet radiation lamp crosses the bottom wall and is itself elongate in a direction substantially perpendicular to the bottom wall in the intermediate space.

24. The device as claimed in claim 17, wherein the base is fixed in the storage case by reversible fixing means allowing it to be released and removed from the storage case.

25. The device as claimed in claim 24, wherein the reversible fixing means comprise slide means allowing the engagement and disengagement of the base by simply moving it in relative translation with respect to the storage case.

26. The device as claimed in claim 17, wherein each stabilizing means comprises a peg on which a distal-end opening of a blow-dry roller can be fitted and retained.

27. The device as claimed in claim 26, wherein said peg has a cylindrical base portion of circular cross section that is joined to a tapered free-end portion.

28. The device as claimed in claim 26, wherein the stabilizing means are designed to receive and retain any blow-dry roller of said plurality of blow-dry rollers.

29. The device as claimed in claim 17, wherein the electrical connection means comprise an electrical safety switch, which is actuated by the lid and designed to prevent the ultraviolet radiation source from being supplied with power when the lid is not in the closed position.

30. The device as claimed in claim 17, wherein the lid comprises magnets that are able to retain, on its inner face, instruments comprising a ferromagnetic material.

31. An assembly comprising a device as claimed in claim 17, a plurality of blow-dry rollers, and at least one removable gripping handle to which one of the blow-dry rollers can be fixed.

32. The assembly as claimed in claim 31, wherein the blow-dry rollers comprise a cylindrical cage provided at a proximal end with a fixing end piece that is able to receive and retain, by axial engagement and in a removable manner, a fixing portion of the gripping handle.
